(19) United States
(12) Patent Application Publication
(10) Pub. No.: US 2026/0306157 A1
(43) Pub. Date: Oct. 8, 2026

(54) FLOW DEVICE FOR SURGICAL SMOKE EVACUATING SYSTEMS

(71) Applicant: Covidien LP, Mansfield, MA (US)

(72) Inventors: Jacob C. Baril, Norwalk, CT (US); Saumya Banerjee, Southington, CT (US); Garrett P. Ebersole, Hamden, CT (US)

(21) Appl. No.: 18/998,289

(22) PCT Filed: Aug. 2, 2023
(86) PCT No.: PCT/IB2023/057841

§ 371 Date: Jan. 24, 2025

Related U.S. Application Data
(60) Provisional application No. 63/396,245, filed on Aug. 9, 2022.

(51) Int. Cl. A61B 18/00; A61B 90/00

(52) U.S. Cl. CPC A61B 18/00; A61B 90/08; A61B 2090/08021; A61B 2218/006; A61B 2218/008

(57) ABSTRACT

A flow device for a smoke evacuating system includes a housing, an inlet, a first outlet, a second outlet, and a spindle assembly. The housing has a longitudinal axis and defines a first chamber and a second chamber. The inlet and the outlet are in fluid communication with the first chamber of the housing. The second outlet is in fluid communication with the second chamber of the housing. The spindle assembly includes a first fan and a second fan and is configured to rotate about the longitudinal axis of the housing.

### FIELD

The present technology relates to tissue removal and, more particularly, to tissue guards and systems incorporating the same for use in tissue removal procedures requiring surgical smoke evacuation.

### BACKGROUND

In minimally-invasive surgical procedures, operations are carried out within an internal body cavity through small entrance openings in the body. The entrance openings may be natural passageways of the body or may be surgically created, for example, by making a small incision into which a cannula is inserted.

Minimally-invasive surgical procedures may be used for partial or total removal of tissue from an internal body cavity. However, the restricted access provided by minimally-invasive openings (natural passageways and/or surgically created openings) presents challenges with respect to maneuverability and visualization. The restricted access also presents challenges when large tissue specimens are required to be removed. As such, tissue specimens that are deemed too large for intact removal may be broken down into a plurality of smaller pieces to facilitate removal from the internal body cavity. When severing tissue or otherwise treating the tissue, surgical smoke may be generated, which may impair visualization and/or otherwise hinder a clinician.

### SUMMARY

As used herein, the term “distal” refers to the portion that is described which is further from a user, while the term “proximal” refers to the portion that is being described which is closer to a user. As used herein, the terms parallel and perpendicular are understood to include relative configurations that are substantially parallel and substantially perpendicular up to about + or −15 degrees from true parallel and true perpendicular.

Further, any or all of the aspects described herein, to the extent consistent, may be used in conjunction with any or all of the other aspects described herein.

Provided in accordance with aspects of the present disclosure is a flow device for a smoke evacuating system. The flow device includes a housing, an inlet, a first outlet, a second outlet, and a spindle assembly. The housing has a longitudinal axis and defines a first chamber and a second chamber. The inlet and the outlet are in fluid communication with the first chamber of the housing. The second outlet is in fluid communication with the second chamber of the housing. The spindle assembly includes a first fan and a second fan and is configured to rotate about the longitudinal axis of the housing.

In an aspect, the housing defines an opening configured to enable a flow of ambient air into the second chamber of the housing.

In an aspect, rotation of the first fan in the first chamber causes corresponding rotation of the second fan in the second chamber.

In an aspect, the first outlet is configured to couple to a vacuum source and the inlet is configured to couple to a tissue guard.

In an aspect, negative airflow applied to the first outlet causes positive airflow to flow from the second outlet.

In accordance with another aspect of the disclosure, a smoke evacuating system includes a tissue guard and a flow device. The tissue guard includes a tubular body, a collar, and an outlet and an inlet extending from the collar. The tubular body has an outwardly flared proximal end portion and a distal end portion and defines a longitudinally-extending passageway. The collar extends from the proximal end portion of the tubular body and defines an annular channel. The inlet defines an inner channel in fluid communication with the annular channel and the outlet defines an inner channel in fluid communication with the annular channel. The flow device is operably coupled to the inlet and the outlet of the tissue guard and is configured to provide positive airflow to the inlet of the tissue guard.

In an aspect, the tissue guard further includes a plurality of hooks coupled to an outer annular section of the collar and adapted to connect to a proximal rim of an access device.

In an aspect, the distal end portion of the tubular body is flared outwardly and includes a plurality of segments that taper in a distal direction, such that adjacent segments of the plurality of segments define a gap therebetween that increases in width in a distal direction.

In an aspect, the collar, the tubular body, the inlet, and the outlet are monolithically formed with one another as a single component.

In an aspect, the flow device includes a first outlet configured to couple to a vacuum source, a second outlet configured to couple to the inlet of the tissue guard, and an inlet configured to couple to the outlet of the tissue guard.

In an aspect, the flow device includes a housing, an inlet, a first outlet, a second outlet, and a spindle assembly. The housing has a longitudinal axis and defines a first chamber and a second chamber. The inlet and the first outlet are in fluid communication with the first chamber. The second outlet is in fluid communication with the second chamber. The spindle includes a first fan and a second fan and is configured to rotate about the longitudinal axis of the housing.

In an aspect, the housing defines an opening configured to enable a flow of ambient air into the second chamber of the housing.

In an aspect, rotation of the first fan in the first chamber causes corresponding rotation of the second fan in the second chamber.

In an aspect, negative airflow applied to the first outlet causes positive airflow to flow from the second outlet.

In accordance with another aspect of the disclosure, a smoke evacuating system includes a tissue guard and a flow device. The tissue guard includes a tubular body, a collar, and an outlet and an inlet extending from the collar. The tubular body has an outwardly flared proximal end portion and a distal end portion and defines a longitudinally-extending passageway. The collar extends from the proximal end portion of the tubular body and defines an annular channel. The inlet defines an inner channel in fluid communication with the annular channel and the outlet defines an inner channel in fluid communication with the annular channel. The flow device includes an inlet, a first outlet, and a second outlet. The inlet is configured to operably couple to the outlet of the tissue guard. The first outlet is in fluid communication with the inlet of the flow device and is configured to operably couple to a vacuum source. The second outlet is configured to operably couple to the inlet of the tissue guard and is configured to provide positive airflow to the inlet of the tissue guard.

In an aspect, the flow device includes a housing having a longitudinal axis and defining a first chamber and a second chamber, and a spindle assembly including a first fan and a second fan. The spindle assembly is configured to rotate about the longitudinal axis of the housing.

In an aspect, the first fan is disposed in the first chamber and the second fan is disposed in the second chamber.

In an aspect, the housing defines an opening configured to enable a flow of ambient air into the second chamber of the housing.

In an aspect, rotation of the first fan in the first chamber causes corresponding rotation of the second fan in the second chamber.

In an aspect, negative airflow applied to the first outlet causes positive airflow to flow from the second outlet.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a perspective view illustrating a smoke evacuating system provided in accordance with aspects of the disclosure;

FIG. 2 is a perspective view illustrating a smoke evacuating tissue guard of the smoke evacuating system of FIG. 1;

FIG. 3 is a longitudinal cross-sectional view illustrating the smoke evacuating tissue guard of FIG. 2;

FIG. 4 is an exploded, top, perspective view of a system provided in accordance with the present disclosure including an access device and the smoke evacuating tissue guard of FIG. 2;

FIG. 5 is a top, perspective view of the system of FIG. 4 in an assembled state;

FIG. 6 is a perspective view of a flow device of the smoke evacuating system of FIG. 1; and

FIG. 7 is a cross-sectional view of the flow device of FIG. 6.

### DETAILED DESCRIPTION

When using a bipolar electrosurgical pencil to dissect a tissue specimen for extraction, a significant amount of smoke plume may be generated. The smoke plume may be harmful to the surgeon and may obstruct the surgical field. The present disclosure provides a smoke evacuating, tissue-cutting guard and a flow device for use with a tissue guard designed to remove smoke from the surgical site as rapidly and as efficiently as possible. The tissue guard may implement unique principles taken from the Venturi Flow Equation to maximize the air evacuated while minimizing the noise produced via turbulence. The flow device may be coupled in-line, between the tissue guard and a vacuum source, to generate a positive pressure airflow into the tissue guard to ensure smoke plume travels from the surgical site and into the suction port(s) of the tissue guard. Further, the tissue guard may prevent smoke accumulation in a specimen bag as well as above the surgical site by evacuating smoke from both areas. The tissue guard may have a relatively large distal opening and a proximal end having an inner diameter sized to shift the pressure/volume ratio. The pressure differential at a necked-down section of the tissue guard forces a mixing or multi-directional pull of the system.

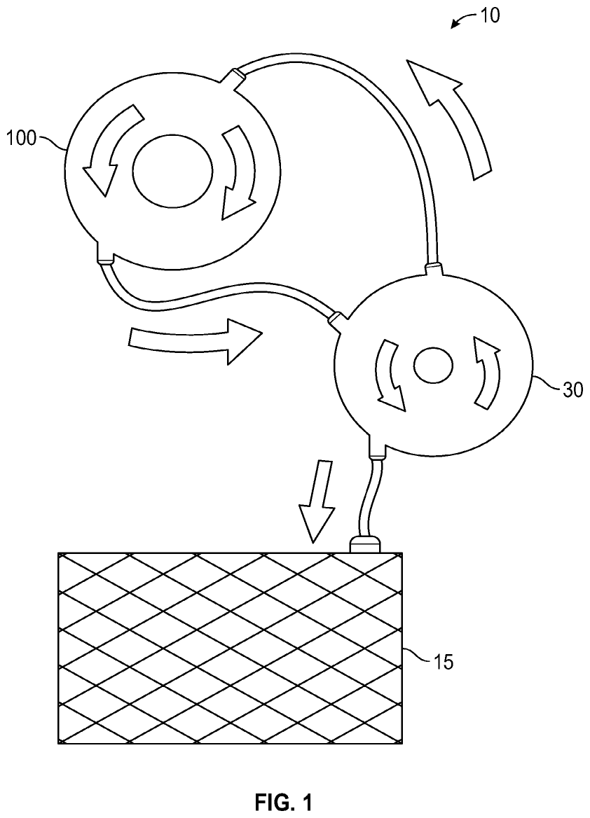

Referring to FIG. 1, a smoke evacuating system is shown and described as smoke evacuating system 10. Smoke evacuating system 10 includes a vacuum source 15 configured to generate suction flow, a flow device 30, and a smoke evacuating tissue guard 100 (also referred to herein as “tissue guard 100”) operably coupled to the vacuum source 15 via the flow device 30.

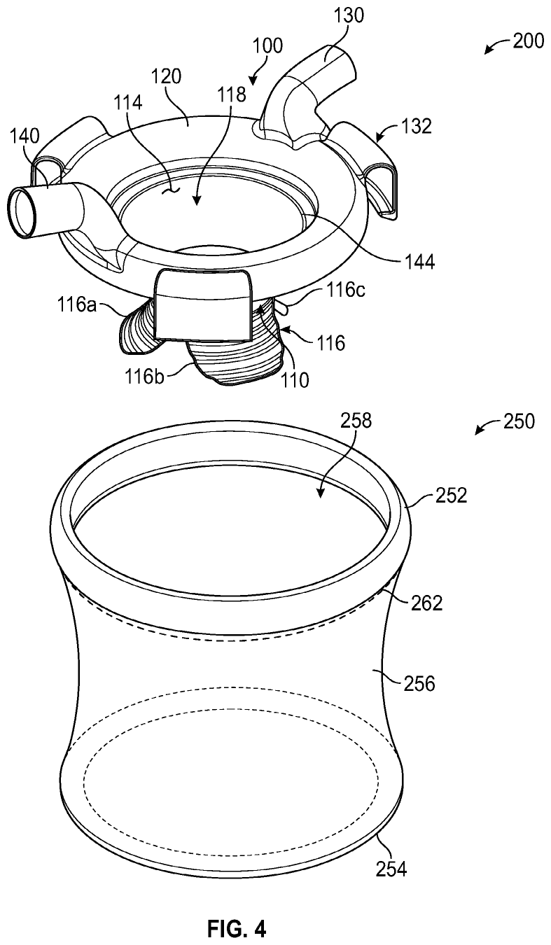

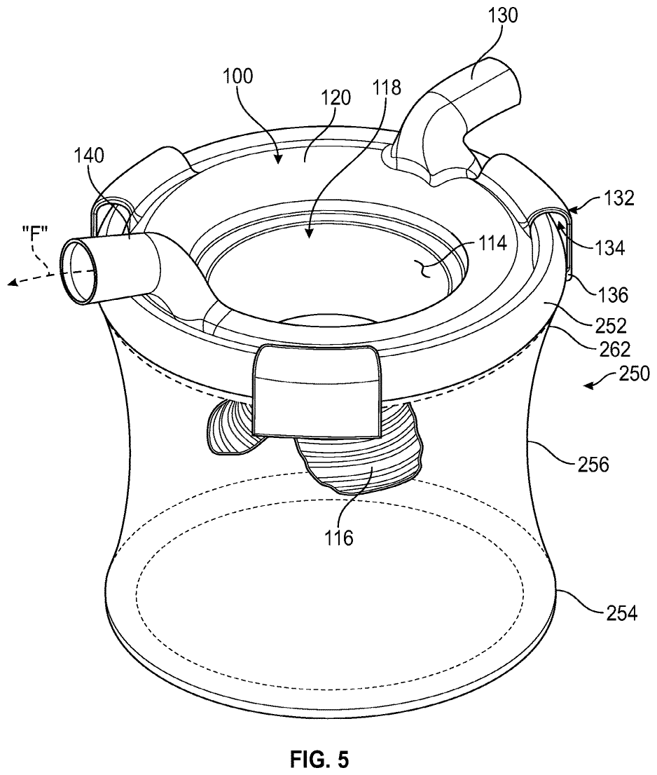

Smoke evacuating tissue guard 100 is formed from a suitable material, e.g., a biocompatible plastic such as, for example, polyethylene, polycarbonate, etc., by any suitable method, e.g., injection molding a single component or multiple components permanently secured or releasably engagable with one another. The material, thickness, and configuration of smoke evacuating tissue guard 100 are selected such that tissue guard 100 defines sufficient stiffness to maintain its shape when positioned within an opening in tissue and/or when engaged within an access device 250 (FIGS. 4 and 5). However, the material, thickness, and configuration of tissue guard 100 also provide sufficient resilient flexibility to permit manipulation of tissue guard 100 from an at-rest position for insertion into an opening in tissue and/or for engagement within access device 250, with tissue guard 100 returning to or towards the at-rest position after insertion and/or engagement. Further, the material, thickness, and configuration of tissue guard 100 is selected such that tissue guard 100 is configured to withstand cutting and puncturing by surgical knives, scalpels, pencils, and the like, thereby protecting surrounding tissue and/or access device 250 from being cut or punctured. Tissue guard 100 may additionally or alternatively be configured to inhibit transfer of thermal and/or electrical energy therethrough to protect surrounding tissue and/or access device 250 from thermal and/or electrical energy.

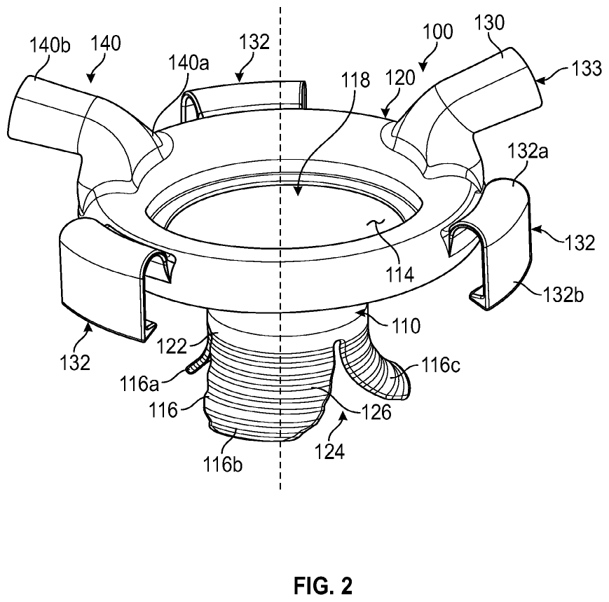

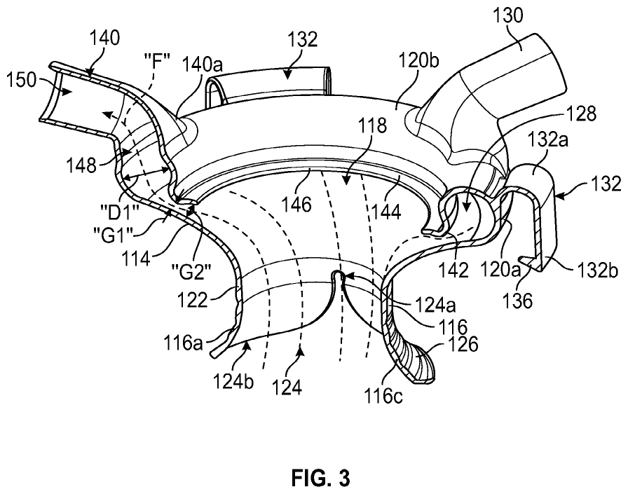

Referring to FIGS. 2 and 3, tissue guard 100 includes a tubular body 110, a collar 120 coupled to a proximal end portion 114 of tubular body 110, and an inlet 130 and an outlet 140 extending from the collar 120 and configured to couple to the vacuum source 15. Tubular body 110, collar 120, inlet 130, and outlet 140 may be monolithically formed as a single component, e.g., via injection molding, or may be distinct attached components. Tubular body 110 defines an open proximal end portion 114, an open distal end portion 116, and a passageway 118 extending between open proximal and distal end portions 114, 116, respectively. Passageway 118 is configured to receive one or more surgical instruments (not shown) therethrough. Proximal and distal end portions 114, 116 of tubular body 110 may each flare outwardly from an intermediate portion 122 of tubular body 110. More specifically, tubular body 110 may have an hourglass shape with a diameter of the open proximal and distal end portions 114, 116 being greater than a diameter of tubular body 110 at intermediate portion 122. In aspects, proximal end portion 114 of tubular body 110 may assume a funnel-shape and may extend at an angle from about 15 degrees to about 50 degrees, and in some aspects about 17 degrees, from a longitudinal axis “X” defined by passageway 118.

Distal end portion 116 of tubular body 110 includes a plurality of segments 116a, 116b, 116c extending distally from intermediate portion 122. Each of segments 116a, 116b, 116c tapers (e.g., gradually narrow) in a distal direction, such that adjacent segments (e.g., segments 116a, 116b) define a cutout or gap 124 therebetween that increases in width in a distal direction. In aspects, cutout 124 defined between adjacent segments 116a, 116b has a rounded proximal end portion 124a whereas a remaining distal end portion 124b of cutout 124 has a generally triangular shape. Other shapes of the cutout 124 are also contemplated, such as, elongated, rectangular, rounded, or the like. In aspects, distal end portion 116 of tubular body 110 may be uninterrupted along its circumference, such that distal end portion 116 is without segments 116a, 116b, 116c or cutouts 124. Cutouts 124 facilitate flexion of distal end portion 116, e.g., to facilitate insertion into an opening in tissue and/or engagement within access device 250 (FIGS. 4 and 5). Each of segments 116a, 116b, 116c has an outer surface having a plurality of horizontally-extending ribs 126 configured to resist backing-out of tissue guard 100 from a surgical opening and/or access device 250 (FIGS. 4 and 5.)

Collar 120 of tissue guard 100 has a ring-shape and defines an annular channel 128 extending circumferentially about passageway 118 of tubular body 110. Annular channel 128 has a circular transverse cross-sectional shape (FIG. 3) having a diameter “D1.” Collar 120 includes an outer annular half-section 120a and an inner annular half-section 120b integrally formed with one another and together defining annular channel 128. Outer annular half-section 120a extends directly from proximal end portion 114 of tubular body 110.

Inner annular half-section 120b of collar 120 extends directly from outer annular half-section 120a and into overlapping alignment with proximal end portion 114 of tubular body 110. Inner annular half-section 120b is spaced from proximal end portion 114 of tubular body 110. More specifically, inner annular half-section 120b has a bent inner edge 142 facing proximal end portion 114 of tubular body 110 to define a first annular gap “G1” therebetween. First annular gap “G1” extends circumferentially about passageway 118 and fluidly interconnects passageway 118 of tubular body 110 and annular channel 128 of collar 120. In aspects, inner annular half-section 120b may define a plurality of oval-shaped openings (not shown) circumferentially spaced from one another along collar 120. The openings are configured to establish fluid communication between passageway 118 and annular channel 128 and to reduce turbulence/noise.

Collar 120 further includes a lip 144 extending radially inward from bent inner edge 142 of inner annular half-section 120b of collar 120. Lip 144 may extend perpendicularly relative to longitudinal axis “X” of passageway 118. In aspects, lip 144 may extend relative to longitudinal axis “X” at any suitable angle. Lip 144 extends circumferentially about passageway 118, thereby defining an outer perimeter of passageway 118. Lip 144 defines a circumferential inner edge 146 that is spaced (in a direction generally parallel with longitudinal axis “X” of passageway 118) from proximal end portion 114 of tubular body 110 to define a second annular gap “G2” therebetween. First annular gap “G1” is sized to be less than diameter “D1” of annular channel 128 of collar 120 and second gap “G2.” By making first annular gap “G1” less than second annular gap “G2” and diameter “D1,” the Venturi Effect dictates that tissue guard 100 establishes a pressure differential in first annular gap “G1” upon application of suction through tissue guard 100, thereby making for a smoother and faster transition from passageway 118 to outlet 140 with minimal disruption of the flow of evacuated smoke.

Collar 120 may further include a plurality of hooks 132 projecting outwardly and ultimately distally from outer annular half-section 120a of collar 120. Each of hooks 132 has a proximal end portion 132a defining an arcuate aperture 134 configured to receive a proximal rim 252 (FIGS. 3 and 4) of access device 250, and a distal end portion 132b having an inwardly-projecting claw 136 configured to engage an undersurface or overhang 262 of proximal rim 252 of access device 250. In aspects, tissue guard 100 may be without hooks 132 and/or hooks 132 may be detachable from collar 120 for instances where tissue guard 100 is to be placed within a tissue opening without utilizing access device 250 (FIGS. 4 and 5).

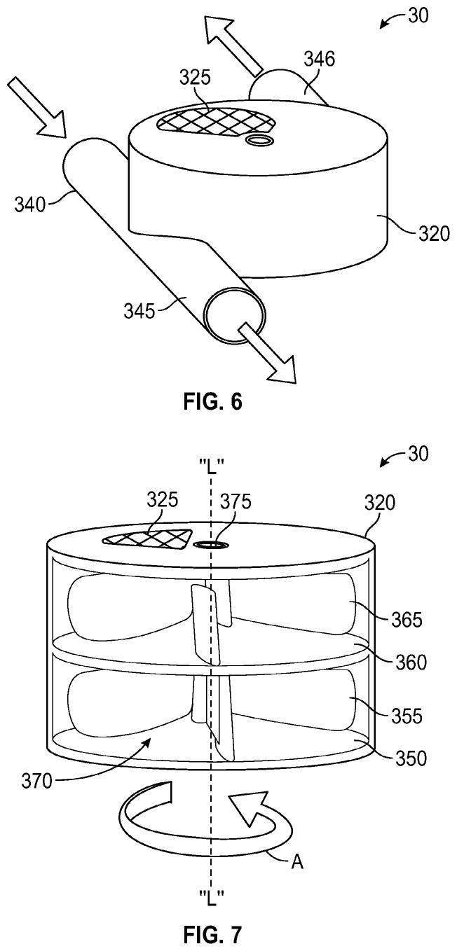

Inlet 130 and outlet 140 of tissue guard 100 define tubular configurations, are formed with outer annular half-section 120a of collar 120, and extend radially outward from collar 120 on opposing ends of collar 120. In aspects, outlet 140 may extend proximally from collar 120 or may assume a helical configuration. Outlet 140 defines an inner channel 150 and has a first end portion 140a in fluid communication, e.g., via an aperture 148 defined through collar 120, with annular channel 128 of collar 120. Outlet 140 has a second end portion 140b configured to connect to an inlet 340 of a flow device (FIG. 6) and ultimately to tubing (not shown) of a vacuum source 15 to enable smoke to be evacuated from an internal surgical site through a fluid path “F” (FIG. 3) that extends from passageway 118 of tubular body 110, into annular channel 128 via second annular gap “G2” and then first annular gap “G1,” and, in turn, into inner channel 150 of outlet 140 via annular channel 128 upon application of suction through smoke evacuating tissue guard 100.

With reference to FIGS. 4 and 5, a system 200 provided in accordance with the present disclosure includes tissue guard 100 and access device 250. Access device 250 may be configured as a tissue retractor, an access port, or other suitable access device configured for positioning within an opening in tissue, e.g., a surgical incision or a naturally-occurring orifice, to provide access therethrough into an internal surgical site. Access device 250 includes proximal rim 252 configured for positioning on an external side of the opening in tissue, a distal rim 254 configured for positioning on an internal side of the opening in tissue, and a body 256 extending between proximal and distal rims 252, 254, respectively. Body 256 is configured to extend through the opening in tissue and defines a passageway 258 extending longitudinally therethrough to permit access to an internal surgical site through the opening in tissue. At least a portion of body 256 of access device 250 may be flexible to facilitate insertion and positioning of access device 250 within the opening in tissue. In aspects, body 256 is formed from a flexible sleeve of material including one or more layers of material. Further, access device 250 may be selectively adjustable, e.g., by rolling proximal rim 254 distally about body 256, to retract tissue and/or secure access device 250 within the opening in tissue. Access device 250 further defines overhang 262 between proximal rim 254 and body 256.

In use, access device 250 is positioned within an opening in tissue such that distal rim 254 is disposed on an internal surface of tissue on the internal side of the opening in tissue, body 256 extends through the opening in tissue, and proximal rim 252 is disposed on an exterior surface of tissue on the external side of the opening in tissue. Access device 250 may be adjusted to conform access device 250 to a patient's anatomy, retracting tissue and/or securing access device 250 within the opening in tissue.

With access device 250 disposed within the opening in tissue, tissue guard 100 is inserted into passageway 258 and hooks 132 of tissue guard 100 are flexed or otherwise manipulated to permit proximal rim 252 to be received in aperture 134 of hooks 132 and claw 136 of hooks 132 to engage overhang 262 of proximal rim 252, thereby locking tissue guard 100 in engagement within access device 250. With tissue guard 100 engaged within access device 250, surgical instrumentation may be inserted through passageway 118 of tubular body 110 of tissue guard 100 into the internal surgical site to, for example, extract a tissue specimen therefrom. Tissue guard 100, as noted above, protects tissue as well as access device 250 during the insertion, manipulation, use and withdrawal of any such surgical instrumentation.

During use, surgical smoke within the surgical site is drawn into passageway 118 of tubular body 110, then into annular channel 128 of collar 120 via first and second annular gaps “G1,” “G2,” then into outlet 140, and then out of a clinician's view. Due to first annular gap “G1” having a reduced area relative to each of an area of second annular gap “G2” and a cross-sectional area of annular channel 128, first annular gap “G1” has a lower relative pressure, thereby making for a smoother and faster transition from passageway 118 to outlet 140 with minimal disruption of the flow of evacuated smoke. In aspects, a ratio of the area of the first annular gap “G1” to the area of the second annular gap “G2” to the cross-sectional area of the annular channel 128 may be about 1:2:4, and in some aspects, about 1:2.1:3.8.

With additional reference to FIGS. 6 and 7, additional airflow may be provided through the tissue guard 100 via the incorporation of an in-line flow device 30, which provides a means of enhancing the overall reduction in smoke plume escaping the tissue guard 100. The flow device 30 optimizes positive and negative airflow to ensure that the maximum volume of smoke plume is captured and filtered out of the surgical setting. The flow device 30 uses the vacuum generated by the vacuum source 15 to power a spindle assembly 370 that provides positive airflow, while also allowing the vacuum to pass through the flow device 30 for removal of smoke plume at the operating site. The additional positive airflow generated by the flow device 30 is directed at the tissue guard 100 towards the suction points in the tissue guard 100, thereby limiting dissipation and escape of smoke plume.

Flow device 30 includes a housing 320 with an inlet 340, a first outlet 345, and a second outlet 346 extending from the housing 320, and a spindle assembly 370 disposed within the housing 320. The inlet 340 of the flow device 30 is configured to operably couple to the outlet 140 of the tissue guard 100 (e.g., directly thereto or via an intermediate tube) and the first outlet 345 is configured to operably couple to the vacuum source 15 (FIG. 1) (e.g., directly thereto or via an intermediate tube). The second outlet 346 is configured to operably couple to the inlet 130 of the tissue guard 100 (e.g., directly thereto or via an intermediate tube).

The housing 320 defines a first chamber 350 and a second chamber 360. The first chamber 350 is in fluid communication with both of the inlet 340 and the first outlet 345 such that airflow may pass from the inlet 340 into the first chamber 350, then from the first chamber 350 to the first outlet 345. The housing 320 also defines an opening 325 enabling ambient air to flow from the outside of the flow device 30 and into the second chamber 360.

The spindle assembly 370 includes a post 375, which is aligned with a longitudinal axis “L” of the flow device 30, and which is rotatable about the longitudinal axis “L” of the flow device 30. The spindle assembly 370 also includes a first fan 355 and a second fan 365 fixed to the post 375 such that rotation of the post 375 about the longitudinal axis “L” (e.g., in the direction of arrow “A”) causes corresponding rotation of the first fan 355 and the second fan 365. The first fan 355 is disposed within the first chamber 350 of the housing 320 and the second fan 365 is disposed within the second chamber 360 of the housing 320.

In use, suction pressure applied by the vacuum source 15 (FIG. 1) to the first outlet 345 causes fluid to flow into the first chamber 350 from the inlet 340 against the blades of the first fan 355, thereby propelling the first fan 355 in the direction of arrow “A” within the first chamber 350. The rotation of the first fan 355 in the first chamber 350 causes rotation of the post 375 about the longitudinal axis “L”, which in turn, causes the second fan 365 to rotate within the second chamber 360. Rotation of the second fan 365 in the second chamber 360 causes ambient air to flow through the opening 325, into the second chamber 360, and from the second chamber 360 to the second outlet 346. From the second outlet 346, the positive airflow pressure is applied to the inlet 130 of the tissue guard 100 and into a portion of the collar 120 (e.g., annular channel 128) to provide additional, positive, airflow thereby ensuring that smoke plume does not escape the tissue guard 100 and assisting the suction of smoke plume through the annular channel 128 and ultimately out of the outlet 140 of the tissue guard 100.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A flow device for a smoke evacuating system, the flow device comprising:
a housing having a longitudinal axis and defining a first chamber and a second chamber;
an inlet and a first outlet in fluid communication with the first chamber;
a second outlet in fluid communication with the second chamber; and
a spindle assembly including a first fan and a second fan, the spindle assembly configured to rotate about the longitudinal axis of the housing.

2. The flow device of claim 1, wherein the housing defines an opening configured to enable a flow of ambient air into the second chamber of the housing.

3. The flow device of claim 1, wherein rotation of the first fan in the first chamber causes corresponding rotation of the second fan in the second chamber.

4. The flow device of claim 1, wherein the first outlet is configured to couple to a vacuum source and the inlet is configured to couple to a tissue guard.

5. The flow device of claim 1, wherein negative airflow applied to the first outlet causes positive airflow to flow from the second outlet.

6. A smoke evacuating system comprising:
a tissue guard including:
a tubular body having an outwardly flared proximal end portion and a distal end portion, the tubular body defining a longitudinally-extending passageway;
a collar extending from the proximal end portion of the tubular body and defining an annular channel;
an outlet extending from the collar and defining an inner channel in fluid communication with the annular channel; and
an inlet extending from the collar and defining an inner channel in fluid communication with the annular channel; and
a flow device operably coupled to the inlet and the outlet of the tissue guard and configured to provide positive airflow to the inlet of the tissue guard.

7. The smoke evacuating system of claim 6, wherein the tissue guard further includes a plurality of hooks coupled to an outer annular section of the collar and adapted to connect to a proximal rim of an access device.

8. The smoke evacuating system of claim 6, wherein the distal end portion of the tubular body is flared outwardly and includes a plurality of segments that taper in a distal direction, such that adjacent segments of the plurality of segments define a gap therebetween that increases in width in a distal direction.

9. The smoke evacuating system of claim 6, wherein the collar, the tubular body, the inlet, and the outlet are monolithically formed with one another as a single component.

10. The smoke evacuating system of claim 6, wherein the flow device includes a first outlet configured to couple to a vacuum source, a second outlet configured to couple to the inlet of the tissue guard, and an inlet configured to couple to the outlet of the tissue guard.

11. The smoke evacuating system of claim 6, wherein the flow device includes:
a housing having a longitudinal axis and defining a first chamber and a second chamber;
an inlet and a first outlet in fluid communication with the first chamber;
a second outlet in fluid communication with the second chamber; and
a spindle assembly including a first fan and a second fan, the spindle assembly configured to rotate about the longitudinal axis of the housing.

12. The smoke evacuating system of claim 11, wherein the housing defines an opening configured to enable a flow of ambient air into the second chamber of the housing.

13. The smoke evacuating system of claim 11, wherein rotation of the first fan in the first chamber causes corresponding rotation of the second fan in the second chamber.

14. The smoke evacuating system of claim 11, wherein negative airflow applied to the first outlet causes positive airflow to flow from the second outlet.

15. A smoke evacuating system comprising:
a tissue guard including:
a tubular body having an outwardly flared proximal end portion and a distal end portion, the tubular body defining a longitudinally-extending passageway;
a collar extending from the proximal end portion of the tubular body and defining an annular channel;
an outlet extending from the collar and defining an inner channel in fluid communication with the annular channel; and
an inlet extending from the collar and defining an inner channel in fluid communication with the annular channel; and
a flow device including:
an inlet configured to operably couple to the outlet of the tissue guard;
a first outlet in fluid communication with the inlet of the flow device and configured to operably couple to a vacuum source; and
a second outlet configured to operably couple to the inlet of the tissue guard and configured to provide positive airflow to the inlet of the tissue guard.

16. The smoke evacuating system of claim 15, wherein the flow device includes:
a housing having a longitudinal axis and defining a first chamber and a second chamber; and
a spindle assembly including a first fan and a second fan, the spindle assembly configured to rotate about the longitudinal axis of the housing.

17. The smoke evacuating system of claim 16, wherein the first fan is disposed in the first chamber and the second fan is disposed in the second chamber.

18. The smoke evacuating system of claim 16, wherein the housing defines an opening configured to enable a flow of ambient air into the second chamber of the housing.

19. The smoke evacuating system of claim 16, wherein rotation of the first fan in the first chamber causes corresponding rotation of the second fan in the second chamber.

20. The smoke evacuating system of claim 15, wherein negative airflow applied to the first outlet causes positive airflow to flow from the second outlet.
